SEQUENCE LISTING (3 sequences)

SEQ ID NO: 1 - DNA, 1659 bp, Artificial Sequence
  Nucleotide sequence of Pre-fusion fusion glycoprotein
atggaactgctgatcctgaaggccaacgccatcaccaccatcctgaccgctgtgaccttctgcttcgccagcggccagaacatcaccgaggaattctaccagagcacctgtagcgccgtgtccaagggctacctgagcgccctgcggaccggctggtacaccagcgtgatcaccatcgagctgagcaacatcaagaaaatcaagtgcaacggcaccgacgccaagatcaagctgatcaagcaggaactggacaagtacaagaacgccgtgaccgagctgcagctgctgatgcagagcacccccgccaccaacaaccaggctagaggcagcggaagcggacggtccctgggcttcctgctgggcgtgggcagcgccattgctagcggagtggccgtgtcaaaggtgctgcacctggaaggcgaagtgaacaagatcaagtccgccctgctgagcaccaacaaggccgtggtgtccctgagcaacggcgtgtccgtgctgaccagcaaggtgctggatctgaagaactacatcgacaagcagctgctgcccatcgtgaacaagcagagctgcagcatccccaacatcgagacagtgatcgagttccagcagaagaacaaccggctgctggaaatcacccgcgagttcagcgtgaacgccggcgtgaccacccccgtgtccacctacatgctgaccaacagcgagctgctgagcctgatcaacgacatgcccatcaccaacgaccagaaaaagctgatgagcaacaacgtgcagatcgtgcggcagcagagctactccatcatgagcatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcgataatgccggcagcgtgtcattctttccacaagccgagacatgcaaggtgcagagcaaccgggtgttctgcgacaccatgaacagcctgaccctgccctccgaagtgaacctgtgcaacgtggacatcttcaaccctaagtacgactgcaagatcatgacctccaagaccgacgtgtccagctccgtgatcacctccctgggcgccatcgtgtcctgctacggcaagaccaagtgcaccgccagcaacaagaaccggggcatcatcaagaccttcagcaacggctgcgactacgtgtccaacaagggggtggacaccgtgtccgtgggcaacaccctgtactacgtgaacaaacaggaaggcaagagcctgtacgtgaagggcgagcccatcatcaacttctacgaccccctggtgttccccagcgaccagttcgacgccagcatcagccaggtcaacgagaagatcaaccagagcctggccttcatcagaaagagcgacgagctgctgcacaatgtgaatgccgtgaagtccaccaccaatatcatgatcaccacaatcatcatcgtgatcatcgtcatcctgctgtccctgatcgccgtgggcctgctgctgtactgcaaggcccggtccacccctgtgaccctgtccaaggaccagctgagcggcatcaacaatatcgccttctccaactga

SEQ ID NO: 2 - DNA, 1014 bp, Artificial Sequence
  Nucleotide sequence of attachment glycoprotein 130-230aa
atggacgccatgaagaggggcctgtgctgcgtgctgctgctgtgcggcgccgtgttcgtgagccccagcaccgtgaaaacaaagaacaccaccaccacccagacccagcccagcaagcccaccaccaagcagcggcagaacaagccccccaacaagcccaacaacgacttccacttcgaggtgttcaacttcgtgccctgcagcatctgcagcaacaaccctacctgctgggccatctgcaagcggaagcctaacaagaagcccggcaagaaaaccaccacaaagcccaccaagaagcctaccttcaagaccacaaagaaggacctgaagccccagaccaccaagcccaaagaggtgcccaccactaagcccggaggcggcggatccacagtgaaaactaagaataccacaacaacacagacacagccttccaagcctacaacaaaacagaggcagaacaaacctcctaacaaacctaacaatgattttcactttgaagtgttcaattttgtgccttgctccatctgctccaacaatccaacatgttgggctatctgtaaacgcaaacccaacaagaaacctgggaaaaagaccaccaccaaacctacaaagaaacccacctttaaaaccaccaagaaagatctgaaacctcagacaacaaaacctaaagaagtgcctactaccaagcccggaggcggcggatccaccgtgaaaacaaaaaacacaacaacaactcagactcagccctctaaacccacaactaagcagagacagaacaagcctccaaacaagccaaacaatgatttccatttcgaagtgtttaactttgtgccatgttctatctgttctaacaatcccacttgttgggccatctgcaaaagaaagccaaacaaaaaacccggcaaaaagacaacaactaagcctaccaaaaagcccacattcaaaactaccaaaaaggatctgaagccacagacaactaagccaaaagaagtgcccacaacaaaaccctgataa

SEQ ID NO: 3 - DNA, 2736 bp, Artificial Sequence
  Nucleotide sequence of Pre-fusion fusion glycoprotein and attachment glycoprotein 130-230aa
atggaactgctgatcctgaaggccaacgccatcaccaccatcctgaccgctgtgaccttctgcttcgccagcggccagaacatcaccgaggaattctaccagagcacctgtagcgccgtgtccaagggctacctgagcgccctgcggaccggctggtacaccagcgtgatcaccatcgagctgagcaacatcaagaaaatcaagtgcaacggcaccgacgccaagatcaagctgatcaagcaggaactggacaagtacaagaacgccgtgaccgagctgcagctgctgatgcagagcacccccgccaccaacaaccaggctagaggcagcggaagcggacggtccctgggcttcctgctgggcgtgggcagcgccattgctagcggagtggccgtgtcaaaggtgctgcacctggaaggcgaagtgaacaagatcaagtccgccctgctgagcaccaacaaggccgtggtgtccctgagcaacggcgtgtccgtgctgaccagcaaggtgctggatctgaagaactacatcgacaagcagctgctgcccatcgtgaacaagcagagctgcagcatccccaacatcgagacagtgatcgagttccagcagaagaacaaccggctgctggaaatcacccgcgagttcagcgtgaacgccggcgtgaccacccccgtgtccacctacatgctgaccaacagcgagctgctgagcctgatcaacgacatgcccatcaccaacgaccagaaaaagctgatgagcaacaacgtgcagatcgtgcggcagcagagctactccatcatgagcatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcgataatgccggcagcgtgtcattctttccacaagccgagacatgcaaggtgcagagcaaccgggtgttctgcgacaccatgaacagcctgaccctgccctccgaagtgaacctgtgcaacgtggacatcttcaaccctaagtacgactgcaagatcatgacctccaagaccgacgtgtccagctccgtgatcacctccctgggcgccatcgtgtcctgctacggcaagaccaagtgcaccgccagcaacaagaaccggggcatcatcaagaccttcagcaacggctgcgactacgtgtccaacaagggggtggacaccgtgtccgtgggcaacaccctgtactacgtgaacaaacaggaaggcaagagcctgtacgtgaagggcgagcccatcatcaacttctacgaccccctggtgttccccagcgaccagttcgacgccagcatcagccaggtcaacgagaagatcaaccagagcctggccttcatcagaaagagcgacgagctgctgcacaatgtgaatgccgtgaagtccaccaccaatatcatgatcaccacaatcatcatcgtgatcatcgtcatcctgctgtccctgatcgccgtgggcctgctgctgtactgcaaggcccggtccacccctgtgaccctgtccaaggaccagctgagcggcatcaacaatatcgccttctccaacgagggccgcggcagcctgctgacctgcggcgacgtggaggagaaccccggccccggtaccgccaccatggacgccatgaagaggggcctgtgctgcgtgctgctgctgtgcggcgccgtgttcgtgagccccagcaccgtgaaaacaaagaacaccaccaccacccagacccagcccagcaagcccaccaccaagcagcggcagaacaagccccccaacaagcccaacaacgacttccacttcgaggtgttcaacttcgtgccctgcagcatctgcagcaacaaccctacctgctgggccatctgcaagcggaagcctaacaagaagcccggcaagaaaaccaccacaaagcccaccaagaagcctaccttcaagaccacaaagaaggacctgaagccccagaccaccaagcccaaagaggtgcccaccactaagcccggaggcggcggatccacagtgaaaactaagaataccacaacaacacagacacagccttccaagcctacaacaaaacagaggcagaacaaacctcctaacaaacctaacaatgattttcactttgaagtgttcaattttgtgccttgctccatctgctccaacaatccaacatgttgggctatctgtaaacgcaaacccaacaagaaacctgggaaaaagaccaccaccaaacctacaaagaaacccacctttaaaaccaccaagaaagatctgaaacctcagacaacaaaacctaaagaagtgcctactaccaagcccggaggcggcggatccaccgtgaaaacaaaaaacacaacaacaactcagactcagccctctaaacccacaactaagcagagacagaacaagcctccaaacaagccaaacaatgatttccatttcgaagtgtttaactttgtgccatgttctatctgttctaacaatcccacttgttgggccatctgcaaaagaaagccaaacaaaaaacccggcaaaaagacaacaactaagcctaccaaaaagcccacattcaaaactaccaaaaaggatctgaagccacagacaactaagccaaaagaagtgcccacaacaaaaccctgataa

(19) United States
(12) Patent Application Publication
(10) Pub. No.: US 2024/0091334 A1
(43) Pub. Date: Mar. 21, 2024

(54) COMBINED VACCINE AGAINST HUMAN RESPIRATORY SYNCYTIAL VIRUS (RSV) INFECTIONS AND METHOD THEREOF FOR INDUCING IMMUNE RESPONSE

(71) Applicants: BEIJING JIAOTONG UNIVERSITY, Beijing (CN); BEIJING ZHIFEI LVZHU BIOPHARMACEUTICAL CO., LTD., Beijing (CN)

(72) Inventors: Jinsheng HE, Beijing (CN); Lin DU, Beijing (CN); Yuanhui FU, Beijing (CN); Weihua ZHU, Beijing (CN); Xianglei PENG, Beijing (CN); Bo GAO, Beijing (CN); Yanpeng ZHENG, Beijing (CN); Meiqin LIU, Beijing (CN)

(21) Appl. No.: 17/768,074

(22) PCT Filed: Aug. 11, 2021
(86) PCT No.: PCT/CN2021/111960

§ 371 Date: Apr. 11, 2022

(30) Foreign Application Priority Data
Aug. 25, 2020 (CN) 202010863764.9

(51) Int. Cl. A61K 39/12; A61P 31/14; A61P 37/04

(52) U.S. Cl. CPC A61K 39/12; A61P 31/14; A61P 37/04; A61K 2039/5256

(57) ABSTRACT

The present disclosure provides a combined vaccine against human respiratory syncytial virus (RSV) infection and a method thereof for inducing immune response. The combined vaccine includes; a first composition including an immunologically effective dosage of a replication-deficient human adenovirus type 26 vector contains a nucleotide encoding an antigenic protein of RSV and a pharmaceutically acceptable vector; and a second composition including an immunologically effective dosage of a replication-deficient chimpanzee adenovirus type 63 vector contains a nucleotide encoding an antigenic protein of RSV and a pharmaceutically acceptable vector. The first composition is a primary immunization composition and the second composition is a booster immunization composition, or vice versa. In the present disclosure, the combined vaccine is used for inducing a protective immunity against RSV infection, and a method is provided for generating the protective immunity against RSV infection.

### CROSS REFERENCE TO RELATED APPLICATION(S)

This application claims the benefit and priority of Chinese patent application No. 202010863764.9 filed before China National Intellectual Property Administration on Aug. 25, 2020 and entitled “COMBINED VACCINE AGAINST RESPIRATORY SYNCYTIAL VIRUS (RSV) INFECTIONS AND METHOD THEREOF FOR INDUCING IMMUNE RESPONSE”, which is incorporated by reference herein in its entirety.

### TECHNICAL FIELD

The present disclosure relates to the technical field of bioengineering, in particular to a combined vaccine against human respiratory syncytial virus (RSV) infection and a method thereof for inducing immune response.

### BACKGROUND ART

Human respiratory syncytial virus (RSV) is widespread worldwide, and susceptible populations of it are mainly children, the elderly and immunocompromised patients. 70% of infants and young children may be infected with RSV before the age of 1, and almost all infants and young children may be infected with RSV before the age of 2. RSV is the most important pathogen causing pneumonia for children under 5 years old, and is an important pathogen leading to hospitalization and death for the elderly and the immunocompromised patients. About 33.1 million children worldwide are infected with RSV every year, wherein 99% of the children who died are from developing countries. It can be seen that RSV brings a heavy economic and social burden to countries around the world, especially developing countries. However, there is no currently-available commercial RSV vaccine for children.

Adenovirus vectors are widely used as vaccine vectors due to their high replication titer, desirable safety and high-efficiency expression of transgene. Similar to live attenuated vaccines, adenovirus vector vaccines can induce mucosal and cellular immunity; meanwhile, adenovirus vectors also have an adjuvant effect to induce a stronger immune response against transgene encoded protein in the body. However, humans are extensively infected with adenovirus type 5 (Ad5), and pre-existing immunity to the Ad5 can limit the efficacy of recombinant adenovirus type 5 (rAd5) vaccines. The levels of pre-existing antibody against human adenovirus type 26 (Ad26) and chimpanzee adenovirus type 63 (ChAd63), respectively, are lower than that of Ad5, avoiding the poor performance when Ad5 is exploited. Moreover, both recombinant Ad26 (rAd26) and recombinant ChAd63 (rChAd63) can be grown to high titers on cell lines suitable tier production of clinical-grade vaccines.

When adenovirus vector vaccine is used to immunize it can induce the vaccinee to produce an immune response against the adenovirus vector itself, which will interfere with the efficacy of boosting immunization if the same vector is used twice. Therefore, a heterologous prime-boost immunization by using two kinds of different adenovirus vectors is beneficial to avoid an inhibiting effect of an induced adenovirus antibodies through primary immunization OD a homologous booster.

### SUMMARY

In view of this, to overcome the shortcomings of the prior art, an objective of the present disclosure is to provide a combined vaccine against RSV infections and a method thereof for inducing immune response.

To achieve the foregoing objective, the present disclosure adopts the following technical solution.

A combined vaccine against RSV infections, including: a first composition and a second composition; wherein

the first composition includes an immunologically effective dosage of a replication-deficient human adenovirus type 26 vector and a pharmaceutically acceptable vector, and the replication-deficient human adenovirus type 26 vector includes a nucleotide encoding an antigenic protein of RSV;

the second composition includes an immunologically effective dosage of a replication-deficient chimpanzee adenovirus type 63 vector and a pharmaceutically acceptable vector, and the replication-deficient chimpanzee adenovirus type 63 vector includes a nucleotide encoding an antigenic protein of RSV;

wherein the first composition is a primary immunization composition and the second composition is a booster immunization composition; alternatively, the first composition is the booster immunization composition, and the second composition is the primary immunization composition.

Preferably, in the first composition, the replication-deficient human adenovirus type 26 vector includes a nucleotide encoding a pre-fusion fusion glycoprotein and/or a nucleotide encoding an adhesion glycoprotein 130-230aa of RSV.

Preferably, in the second composition, the replication-deficient chimpanzee adenovirus type 63 vector includes a nucleotide encoding preF and/or a nucleotide encoding G 130-230.

Preferably, in the first composition, the replication-deficient human adenovirus type 26 vector includes a nucleotide encoding preF, the nucleotide sequence of which is set forth in. SEQ ID NO: 1.

Preferably, in the first composition, the replication-deficient human adenovirus type 26 vector includes a nucleotide encoding G 130-230, the nucleotide sequence of which is set forth in SEQ ID NO: 2.

Preferably, in the first composition, the replication-deficient human adenovirus type 26 vector includes a nucleotide encoding both pre and G 130-230, the nucleotide sequence of which is set forth in SEQ ID NO: 3.

Preferably, in the second composition, the replication-deficient chimpanzee adenovirus type 63 vector includes nucleic. acids encoding at least one of three antigenic proteins of RSV.

Preferably, in the second composition, the replication-deficient chimpanzee adenovirus type 63 vector includes a nucleic acid encoding at least one of three antigenic proteins of RSV, wherein the nucleic acid comprises the nucleotide sequences that are set forth in SEQ ID NO: 1, SEQ ID NO: 2 and SEQ ID NO: 3.

Preferably, in the first composition, the replication-deficient human adenovirus type 26 vector is rAd26; and in the second composition, the replication-deficient chimpanzee adenovirus type 63 vector is rChAd63.

Preferably, the combined vaccine is used to generate a protective immunity against RSV infections; wherein the first immunizer is used to elicit an immune response and the second composition is used to boost the immune response; alternatively, the second immunizer is used to elicit the immune response and the first composition is used to boost the immune response.)

It can be seen from the technical solutions provided by examples of the present disclosure, the combined vaccine against RSV infection is used for inducing a protective immunity against RSV infections, and a composition, a vaccine and a method for providing the protective immunity against RSV infectious.

Additional aspects and advantages of the present disclosure will be partially given in the following description, and become clear in the following description, or be learned through the practice of the present disclosure.

### BRIEF DESCRIPTION OF DRAWINGS

To describe technical solutions of examples in present disclosure more clearly, briefly introductions of drawings to describe the examples is as following. Apparently, the drawings in the following, description are only some examples of the present disclosure, and for those of ordinary skill in the art, other drawings can also be obtained from these drawings without creative efforts.

FIG. 1 shows a preF-specific IgG produced by recombinant adenovirus-immunized BALB/c mice provided in examples of the present disclosure;

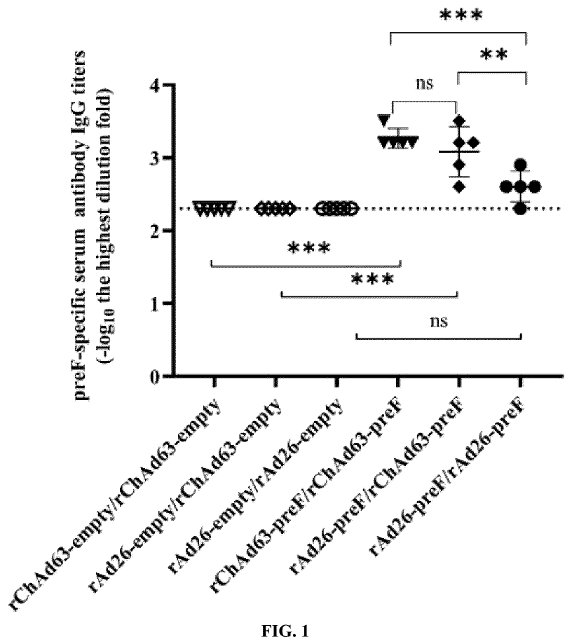

FIG. 2 shows a postF-specific IgG produced by recombinant adenovirus-immunized BALB/c mice provided in examples of the present disclosure:

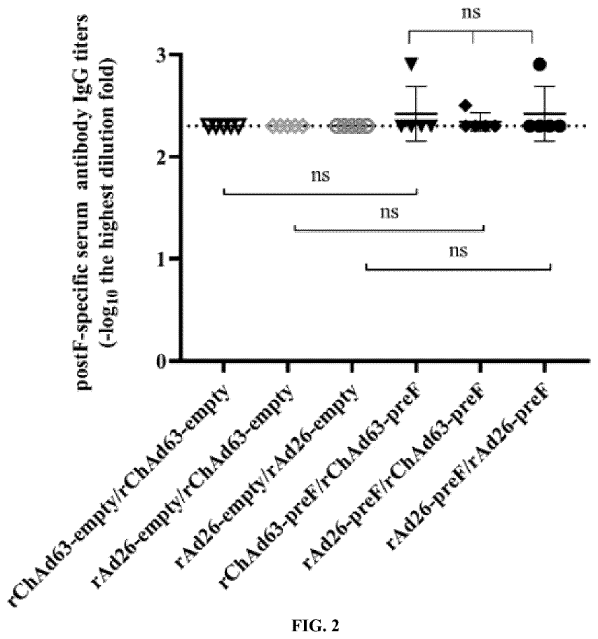

FIG. 3 shows a serum neutralizing antibody produced by recombinant adenovirus-immunized BALB/c mice provided in examples of the present disclosure;

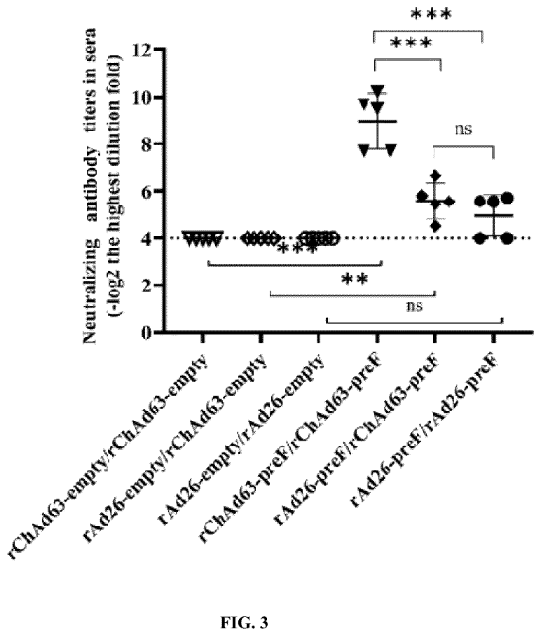

FIG. 4 shows a RSV F-specific CD8+T cell immune response produced by recombinant adenovirus-immunized BALB/c mice provided in examples of the present disclosure;

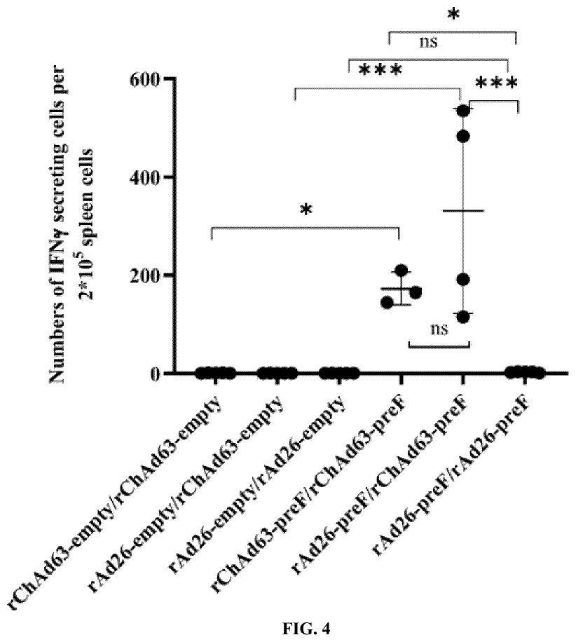

FIG. 5 shows a preF-specific IgG produced by recombinant adenovirus-immunized BALB/c mice provided in examples of the present disclosure;

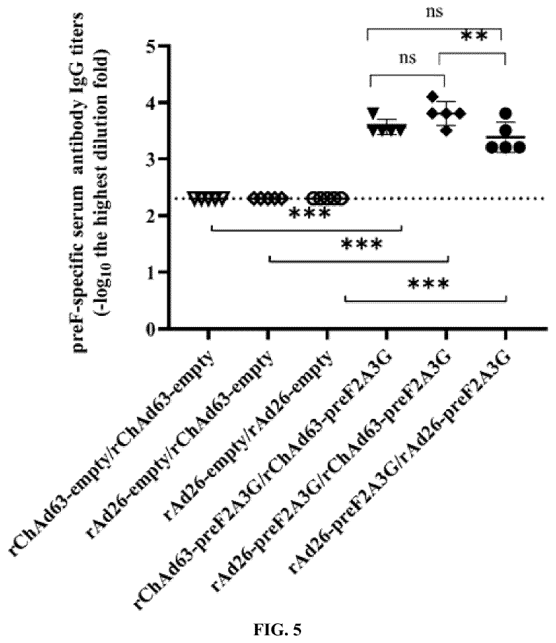

FIG. 6 shows a postF-specific IgG produced by recombinant adenovirus-immunized BALB/c mice provided in examples of the present disclosure:

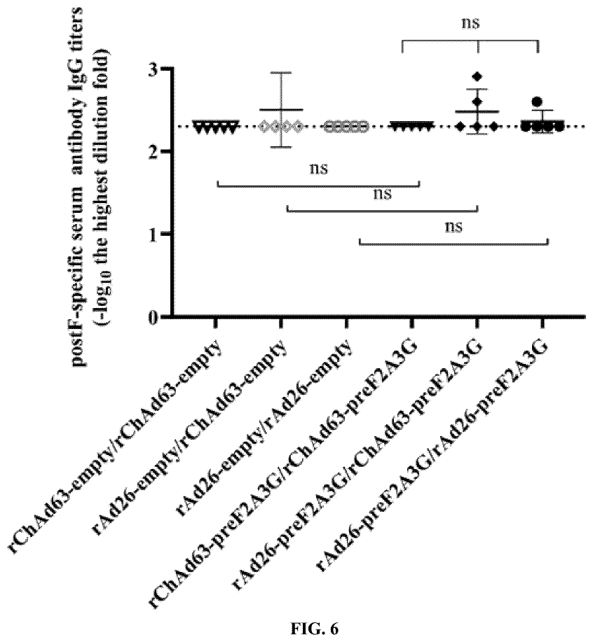

FIG. 7 shows a G-specific IgG produced by recombinant adenovirus-immunized BALB/c mice provided in examples of the present disclosure;

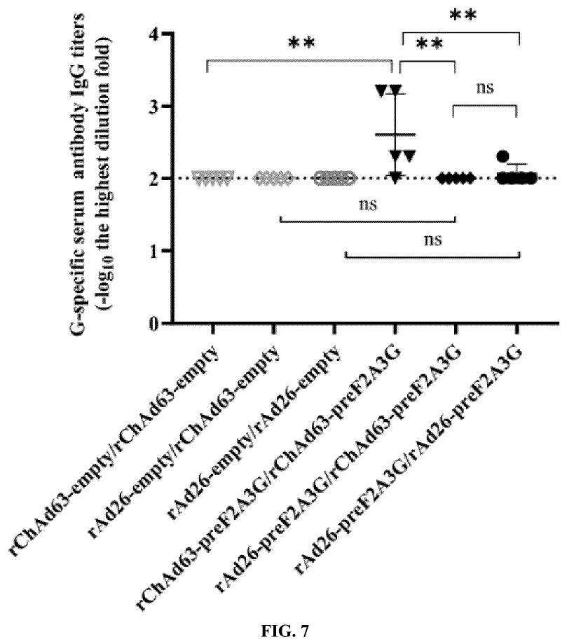

FIG. 8 shows a serum neutralizing antibody produced by recombinant adenovirus-immunized BALB/c mice provided in examples of the present disclosure: and

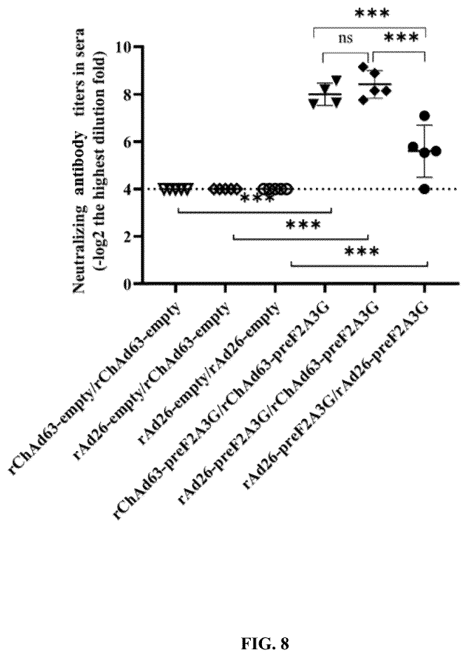

FIG. 9 shows a RSV F-specific CD8+T immune response produced by recombinant adenovirus-immunized BALB/c mice provided in examples of the present disclosure.

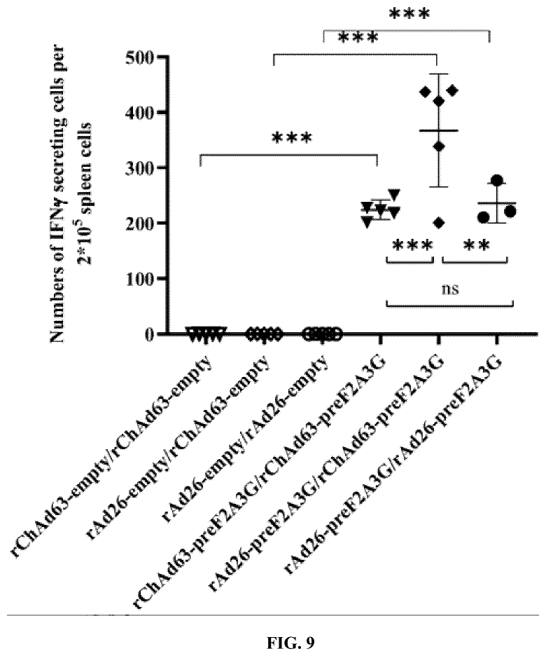

### DETAILED DESCRIPTION OF THE EMBODIMENTS

Embodiments of the present disclosure are described below in detail. Examples of the embodiments are shown in drawings. The same or similar numerals represent the same or similar elements or elements having the same or similar functions throughout the disclosure. Embodiments described below with reference to drawings are examples, and are only used to explain the present disclosure but should not he construed as a limitation herein.

Those skilled in the art can understand that, unless otherwise stated, the singular forms “a”, “an”, “said” and “the” used herein may also include plural forms. It should be further understood that the word “comprising” or “including” used in the specification of the present disclosure refers to presence of the described features, integers, steps, operations, elements and/or components, but does not exclude presence or addition of one or more other features, integers, steps, operations, elements, components, and/or groups thereof. It should he understood that when an element is “connected” or “coupled” to another element, it can be connected or coupled to another element directly or through an intermediate element, in addition, “connected” or “coupled” used herein can include wireless connection or coupling. Term “and/or” used herein includes any unit and all combinations of one or more of the associated listed items.

Those skilled in the art can understand that, unless otherwise defined, all terms (including technical terms and scientific terms) used herein have the same meanings as those commonly understood by those of ordinary skill in the art herein. It should also be understood that terms such as those defined in general dictionaries should be understood as having meanings consistent with the meanings in the context of the prior art, and unless otherwise defined herein, are not be explained in ideal or overly-formal meanings.

For ease of understanding of the examples herein, further explanation and description is given by taking several specific examples as examples in conjunction with drawings, and each example does not constitute a limitation to the examples herein.

The present disclosure aims to explore protective immunity against RSV infections by a prime-boost immunization strategy, specifically a composition, a vaccine and a method for the protective immunity against RSV infections.

It is found that a prime-boost combination of a replication-deficient adenovirus vector vaccine produces an effective immune protection against RSV. Therefore, examples of the present disclosure provide a combined vaccine against RSV infections, including: a first composition and a second composition; wherein

the first composition includes an immunologically effective dosage of a replication-deficient human adenovirus type 26 vector and a pharmaceutically acceptable vector, and the replication-deficient human adenovirus type 26 vector includes a nucleotide encoding an antigenic protein of RSV;

the second composition includes an immunologically effective dosage of a replication-deficient chimpanzee adenovirus type 63 vector and a pharmaceutically acceptable vector, and the replication-deficient chimpanzee adenovirus type 63 vector includes a nucleotide encoding an antigenic protein of RSV.

In one embodiment of the present disclosure, the first composition is a primary immunization composition and the second composition is a booster immunization composition; alternatively, the first composition is a booster immunization composition, and the second composition is a primary immunization composition.

In one example of the present disclosure, in the first composition, the replication-deficient human adenovirus type 26 vector includes: a nucleotide encoding a pre-fusion fusion glycoprotein (preF) and/or a nucleotide encoding an attachment glycoprotein 30-230aa (G 130-2301 of RSV; wherein the nucleotide encoding the attachment glycoprotein 30-230aa of RSV is abbreviated as: preF2A3G.

In the second composition, the replication-deficient chimpanzee adenovirus type 63 vector includes: a nucleotide encoding preF and/or a nucleotide encoding G 130-230.

In one example of the present disclosure, in the first composition, the replication-deficient human adenovirus type 26 vector includes a nucleotide encoding preF, the nucleotide sequence of which is set forth in SEQ ID NO: 1; or a nucleotide encoding, G 130-230, the nucleotide sequence of which is set forth in SEQ ID NO: 2; or a nucleotide encoding both preF and G 130-230, the nucleotide sequence of which is set forth in SEQ ID NO: 3.

In one example of the present disclosure, in the second composition, the replication-deficient chimpanzee adenovirus type 63 vector includes a nucleic acid encoding at least one of three antigenic proteins of RSV. In particular, the a nucleic acid encoding at least one of three antigenic proteins of RSV, wherein the nucleic acid comprises the nucleotide sequences that are set forth in SEQ ID NO: 1, SEQ ID NO: 2 and SEQ ID NO: 3.

In one example of the present disclosure, in the first composition, the replication-deficient human adenovirus type 26 vector is rAd26; and in the second composition, the replication-deficient chimpanzee adenovirus type 63 vector is rChAd63.

The examples of the present disclosure further provide a method for inducing an immune response against RSV in the vaccinee, including the following steps:

A. administering the first composition including an immunologically effective amount of an adenovirus vector to a subject, wherein the adenovirus vector includes a nucleotide encoding an antigenic protein of RSV; and

B. administering the second composition including an immunologically effective amount of an adenovirus vector to the subject, wherein the adenovirus vector includes a nucleotide encoding the antigenic protein of RSV; wherein

step (A) and step (B) are conducted in any order.

### EXAMPLE 1

Prime-boost immunization of a recombinant adenovirus encoding a pre-fusion fusion glycoprotein (preF) of RSV

### I. Animal Immunity

Female BALB/c mice aged 6-8 weeks were divided into 6 groups; on day 0, a basal serum was collected; on day 1, first immunization was conducted by intramuscular injection. On the 21st day after immunization, blood was collected after the first immunization. On the 28th day, second immunization was conducted by intramuscular injection. On the 49th day, blood was collected alter the second immunization. On the 56th day, a challenge experiment was conducted via an intranasal route, and a challenge dosage was 1×10⁶ pfu/50 μl of [symbol] RSV.

Grouping and processing were as follows:

a first group (group G1): secondary immunization was conducted by intramuscular injection; wherein a primary immunizer was an rChAd63/empty virus solution (with a viral load of 1×10¹⁰ vp), and a secondary immunizer was an rChAd63/empty virus solution (with a viral load of 1×10¹⁰ vp);

a second group (group G2): secondary immunization was conducted by intramuscular injection; wherein a primary immunizer was an rAd26/empty virus solution (with a viral load of 1×10¹⁰ vp), and a secondary immunizer was an rAd63/empty virus solution (with a viral load of 1×10¹⁰ vp);

a third group (group G3 group): secondary immunization was conducted by intramuscular injection; wherein a primary immunizer was an rAd26/empty virus solution (with a viral load of 1×10¹⁰ vp), and a secondary immunizer was an rChAd26/empty virus solution (with a viral load of 1×10¹⁰ vp);

a fourth group (group G4): secondary immunization was conducted by intramuscular injection; wherein a primary immunizer was an rChAd63/preF virus solution (with a viral load of 1×10¹⁰ vp), and a secondary immunizer was an rChAd63/preF virus solution (with a viral load of 1×10¹⁰ vp);

a fifth group (group G5): secondary immunization was conducted by intramuscular injection; wherein a primary immunizer was an rAd26/preF virus solution (with a viral load of 1×10¹⁰ vp), and a secondary immunizer was an rAd63/preF virus solution (with a viral load of 1×10¹⁰ vp); and

a sixth group (group C16): secondary immunization was conducted by intramuscular injection; wherein a primary immunizer was an rAd26/preF virus solution (with a viral load of 1×10¹⁰ vp), and a secondary immunizer was an rChAd26/preF virus solution (with a viral load of 1×10¹⁰ vp).

### II. Detection of IgG in Serum of Mice After Immunization

A solution to be tested was: serum isolated and Obtained from venous blood of mice collected on the 49th day after immunization in step I.

A serum antibody titer was detected in mice by enzyme-linked immunosorbent assay (ELISA). Purified RSV was coated on a microtiter plate at 2,500 pfu/well, a pre-fusion F protein (preF) was coated on a microtiter plate at 450 ng/well, and a post-fusion F protein (postF) was coated on a microliter plate at 1,200 ng/well, and serum antibody IgG in mice was detected by ELISA.

### III. Serum Neutralizing Antibodies of Immunized Mice

1. HEp-2 cells were inoculated onto a 96-well plate at density of 2.0×10⁴ cells/well, and cultured for 24 h.

2. Serum of the immunized mice was inactivated at 56° C. for 30 min, and diluted twice with a Dulbecco's Modified Eagle Medium (DMEM) medium containing 2% fetal bovine serum. RSV-mGFP was added to the sera of different dilutions with a final concentration to 1000 PFU/100 μl, and the sera were mixed then incubated at 37° C. for 1 h.

3. A medium of the 96-well plate was discarded, the plate was washed 1-2 times with PBS, and 100 μl of a mixture (containing 1000 PFU of RSV-mGFP) was added to the 96-well plate plated with a monolayer of HEp-2 cells, followed by incubation at 37° C. for 48 h. At the same time, a blank control group (no virus, no serum or antibody), a negative control group (with RSV-mGFP, no serum) and a positive control group (RSV polyclonal antibody alternative serum) were set up, and cultured for 2 d.

4. After completing step 3, a fluorescence intensity of green fluorescent proteins in each well was detected using a multifunctional microplate reader SpectraMax M5e at an excitation wavelength 479 nm and an emission wavelength 517 nm for 10 sec.

5. The blank control group was subtracted from the experimental group, the negative control group and the positive control group, respectively, and a linear regression equation of the experimental group was established by SPSS: an antibody titer with a 50% reduction in fluorescence intensity (IC₅₀) was calculated by the linear regression equation.

### IV. Analysis of an Cellular Immune Effect of Immunized Mice

Cells to be tested were: spleen cells of mice oft the 49th day after immunization in step I.

1. Mice of 49 d after immunization were sacrificed by cervical dislocation, and spleen cells were collected aseptically in an ultra-clean bench.

2, An effect of cellular immunity was analyzed by an enzyme-linked immunobsorbent spot (ELISPOT) method, and a number of spleen lymphocytes secreting IFN-γ after stimulation with H-2K d-restricted CTL epitope of a RSV F protein was counted.

3. In an ultra-clean bench, 200 μl of a 1640 medium was added to each well, and after standing at room temperature for 5-10 min, the 1640 medium was discarded.

4. A cell suspension was added to each experimental well at 100 μl/well. 2×10⁵ cells/well were added to positive control wells, negative control wells and experimental wells, while no cells were added to background control wells.

5. A volume of stimulus added to each well was 10 μl/well; a positive stimulus working solution was added to positive control wells; a serum-free 1640 medium was added to negative control wells and the background control wells; RSV F protein H-2K d-restricted CTL epitopes (KYKNAVTEL and TYMLTNSELL, purity≥95%) were added to the experimental wells (0.5 μg each of KYKNAVTEL and TYMLTNSELL was added to 2×10⁵ cells); and incubation was conducted in an incubator at 37° C., with 5% CO₂ for 24-48 h.

6. After completing, step 5, cells and medium in the well were poured out, and ice-cold deionized water was added at 200 μl/well; the cells were placed in a refrigerator at 4° C. for 10 min to conduct hypotonic lysis of cells.

7. Liquid in the well was poured out, followed by washing 5-7 times with 1×Washing buffer, at 200 μl/well, each time for 30-60 sec, and dried on an absorbent paper.

8. A diluted and biotin-labeled antibody working solution was added to each experimental well, at 100 μl/well, followed by incubation at 37° C. for 1 h.

9. After completing step 8, liquid in the well was poured out, followed by washing 5-7 times with 1×Washing buffer, at 200 μl/well, each time for 30-60 sec, and dried on an absorbent paper.

10. A diluted and enzyme-labeled avidin working solution was added to each experimental well, at 100 μl/well, followed by incubation at 37° C. for 1 h.

11. Liquid in the well was poured out, followed by washing 5-7 times with 1×Washing buffer, at 200 μl/well, each time for 30-60 sec, and dried on an absorbent paper.

12. A newly-prepared color developer working solution AEC was added to each experimental well, at 100 μl/well, and allowed to stand at room temperature for 15-45 min.

3. After spots had grown to a suitable size, liquid in the well was poured out, a base of a plate was uncovered, and washed with deionized water for 3-5 times to terminate color development; the plate was placed in a cool place at room temperature to dry naturally until it was completely dry; a 96-well plate was imaged with an ELISPOT reader, and a number of spots was read and analyzed with ImmunoSpot image analyzer software v4.0.

### EXAMPLE 2

Prime-boost immunization of a recombinant adenovirus co-expressing a pre-fusion fusion glycoprotein (preF) and an attachment glycoprotein of RSV.

### I. Animal Immunity

Female BALB/c mice aged 6-8 weeks were divided into 6 groups; on day 0, a basal serum was collected; on day 1, first immunization was conducted by intramuscular injection. On the 21st day after immunization, blood was collected after the first immunization. On the 28th day, second immunization was conducted by intramuscular injection. On the 49th day, blood collection was conducted continuously after the second immunization. On the 56th day, a challenge experiment was conducted via an intranasal route, wherein a challenge dosage was 1×10⁶ pfu/50 μl of [symbol] RSV.

### Grouping and Processing Were as Follows:

a first group (group G1): secondary immunization was conducted by intramuscular injection; wherein a primary immunizer was an rChAd63/empty virus solution (with a viral load of 1×10¹⁰ vp), and a secondary immunizer was an rChAd63/empty virus solution with a viral load of 1×10¹⁰ vp);

a second group (group G2): secondary immunization was conducted by intramuscular injection; wherein a primary immunizer was an rAd26/empty virus solution (with a viral load of 1×10¹⁰ vp), and a secondary immunizer was an rChAd63/empty virus solution (with a viral load of 1×10¹⁰ vp);

a third group (group G3): secondary immunization was conducted by intramuscular injection; wherein a primary immunizer was an rAd26/empty virus solution (with a viral load of 1×10¹⁰ vp), and a secondary immunizer was an rChAd26/empty virus solution (with a viral load of 1×10¹⁰ vp);

a fourth group (group G4): secondary immunization was conducted by intramuscular injection; wherein a primary immunizer was an rChAd63/preF2A3G virus solution (with a viral load of 1×10¹⁰ vp), and a secondary immunizer was an rChAd63/preF2A3G virus solution (with a viral load of 1×10¹⁰ vp);

a fifth group (group G5): secondary immunization was conducted by intramuscular injection; wherein a primary immunizer was an rAd26/preF2A3G virus solution (with a viral load of 1×10¹⁰ vp), and a secondary immunizer was an rAd63/preF2A3G virus solution (with a viral load of 1×10¹⁻ vp); and

a sixth group (group G6): secondary immunization was conducted by intramuscular injection; wherein a primary immunizer was an rAd26/preF2A3G virus solution (with a viral load of 1×10¹⁰ vp), and a secondary immunizer was an rChAd26/preF2A3G virus solution (with a viral load of 1×10¹⁰ vp).

II. Detection of IgG in serum of mice after immunization

A solution to be tested was: serum isolated and obtained from venous blood of mice collected on the 49th day after immunization in step I.

A serum antibody titer was detected in mice by enzyme-linked immunosorbent assay (ELISA). Purified RSV was coated on a microtiter plate at 2,500 pfu/well, a pre-fusion F protein (preF) was coated on a microtiter plate at 450 ng/well, the post-fusion F protein (postF) was coated on a microtiter plate at 1,200 ng/well and a G protein was coated on a microtiter plate at 1,600 ng/well, and a serum antibody IgG in mice was detected by ELISA.

### III. Serum Neutralizing Antibodies of Immunized Mice

1. HeP-2 cells were inoculated onto a 96-well plate at a density of 2.0×10⁴ cells/well, and cultured for 24 h.

2. Serum of the immunized mouse was inactivated at 56° C. for 30 min, and diluted twice with a Dulbecco's Modified Eagle Medium (DMEM) medium containing 2% fetal bovine serum. RSV-mGFP was added to the sera of different dilutions with a final concentration to 1000 PFU/100 μl, and the sera were mixed then incubated at 37° C. for 1 h.

3. A medium of the 96-well plate was discarded, the plate was washed 1-2 times with PBS, 100 μl of a mixture (containing 1000 PFU of RSV-mGFP) was added to the 96-well plate plated with a monolayer of the HEp-2 cells, followed by incubation at 37° C. for 48 h. At the same time, a blank control group (no virus, no serum or antibody), a negative control group (with RSV-mGFP, no serum) and a positive control group (RSV polyclonal antibody alternative serum) were set up, and cultured for 2 d.

4. After completing step 3, a fluorescence intensity of green fluorescent proteins in each well was detected using a multifunctional microplate reader SpectraMax M5e at an excitation wavelength 479 nm and an emission wavelength 517 nm for 10 sec.

5. The blank control group was subtracted from the experimental group, the negative control group and the positive control group, respectively, and a linear regression equation of the experimental group was established by SPSS; an antibody titer with a 50% reduction in fluorescence intensity (IC₅₀) was calculated by the linear regression equation.

### IV. Analysis of an Cellular Immune Effect of Immunized Mice

Cells to be tested were: spleen cells of mice on the 49th day after immunization in step I.

1. Mice of 49 d after immunization were sacrificed by cervical dislocation, and spleen cells were collected aseptically in an ultra-clean bench.

2. An effect of cellular immunity was analyzed by an enzyme-linked immunobsorbent spot (ELISPOT) method, and a number of spleen lymphocytes secreting IFN-γ after stimulation with H-2K d-restricted CTL epitope of a RSV F protein was counted.

3. In the ultra-clean bench, 200 μl of a 1640 medium was added to each well, and after standing at room temperature for 5-10 min, the 1640 medium was discarded.

4. A cell suspension was added to each experimental well, at 100 μ/well. 2×10⁵ cells/well were added to positive control wells, negative control wells and experimental wells, while no cells were added to background control wells.

5. A volume of stimulus added to each well was 10 μl/well a positive stimulus working solution was added to the positive control wells; a serum-free 1640 medium was added to the negative control wells and the background control wells; and RSV F protein H-2K d-restricted CTL epitopes (KYKNAVTEL and TYMLTNSELL, purity ≥95%) were added to the experimental wells (0.5 μg each of KYKNAVTEL and TYMLTNSELL was added to 2×10⁵ cells); and incubation was conducted in an incubator at 37° C. with 5% CO₂ for 24-48 h.

6. After completing step 5, cells and medium in the well were poured out, and ice-cold deionized water was added at 200 μl/well; the cells were placed in a refrigerator at 4° C. for 10 min to conduct hypotonic lysis of cells.

7. Liquid in the well was poured out, followed by washing 5-7 times with 1×Washing buffer, at 200 μl/well, each time for 30-60 sec, and dried on an absorbent paper.

8. A diluted and biotin-labeled antibody working solution was added to each experimental well, at 100 μl/well, followed by incubation at 37° C. for 1 h.

9. After completing step 8, liquid in the well was poured out, followed by washing 5-7 times with 1×Washing buffer, at 200 μl/well, each time for 30-60 sec, and dried on an absorbent paper.

10. A diluted and enzyme-labeled avidin working solution was added to each experimental well, at 100 μl/well, followed by incubation at 37° C. for 1 h.

11. Liquid in the well was poured out, followed by washing 5-7 times with 1×Washing buffer, at 200 μl/well, each time for 30-60 sec, and dried on an absorbent paper.

12. A newly-prepared color developer working solution. AEC was added to each experimental well, at 100 μl/well, and allowed to stand at room temperature for 15-45 min.

13. After spots had grown to a suitable size, liquid in the well was poured out, a base of a plate was uncovered, and washed with deionized water for 3-5 times to terminate the color development; the plate was placed in a cool place at room temperature to dry naturally until it was completely dry; the 96-well plate was imaged with an ELISPOT reader, and a number of spots was read and analyzed with ImmunoSpot image analyzer software v4.0.

The above described are merely specific embodiments of the present disclosure, and the protection scope herein is not limited thereto. Any modification or replacement easily conceived by those skilled in the art within the technical scope of the present disclosure should fall within the claimed scope herein. Therefore, the claimed scope of the present disclosure should be subject to the claimed scope of the claims.

1. A combined vaccine against human respiratory syncytial virus (RSV) infection, comprising: a first composition and a second composition, wherein
the first composition comprises an immunologically effective dosage of a replication-deficient human adenovirus type 26 vector and a pharmaceutically acceptable vector, and the replication-deficient human adenovirus type 26 vector comprises a nucleotide encoding an antigenic protein of RSV;
the second composition comprises an immunologically effective dosage of a replication-deficient chimpanzee adenovirus type 63 vector and a pharmaceutically acceptable vector, and the replication-deficient chimpanzee adenovirus type 63 vector comprises a nucleotide encoding an antigenic protein of RSV; and
the first composition is a primary immunization composition and the second composition is a booster immunization composition; alternatively, the first composition is the booster immunization composition, and the second composition is the primary immunization composition.

2. The combined vaccine according to claim 1, wherein in the first composition, the replication-deficient human adenovirus type 26 vector comprises a nucleotide encoding a pre-fusion fusion glycoprotein (preF) and/or a nucleotide encoding an attachment glycoprotein 130-230aa (G 130-230) of RSV.

3. The combined vaccine according to claim 2, wherein in the first composition, the replication-deficient human adenovirus type 26 vector comprises the nucleotide encoding preF, the nucleotide sequence of which is set forth in SEQ ID NO: 1.

4. The combined vaccine according to claim 2, wherein in the first composition, the replication-deficient human adenovirus type 26 vector comprises the nucleotide encoding G 130-230, the nucleotide sequence of which is set forth in SEQ ID NO: 2.

5. The combined vaccine according to claim 2, wherein in the first composition, the replication-deficient human adenovirus type 26 vector comprises a nucleotide encoding both preF and G 130-230 the nucleotide sequence of which is set forth in SEQ ID NO: 3.

6. The combined vaccine according to claim 1, wherein in the second composition, the replication-deficient chimpanzee adenovirus type 63 vector comprises a nucleotide encoding preF and/or a nucleotide encoding G 130-230aa.

7. The combined vaccine according to claim 1, wherein in the second composition, the replication-deficient chimpanzee adenovirus type 63 vector comprises a nucleic acid encoding at least one of three antigenic proteins of RSV.

8. The combined vaccine according to claim 7, wherein the nucleic acid encoding at least three antigenic proteins of RSV, wherein the nucleic acid comprises the nucleotide sequences that are set forth in SEQ ID NO: 1, SEQ ID NO: 2 and SEQ ID NO: 3.

9. The combined vaccine according to claim 1, wherein in the first composition, the replication-deficient human adenovirus type 26 vector is rAd26; and in the second composition, the replication-deficient chimpanzee adenovirus type 63 vector is rChAd63.

10. The combined vaccine according to claim 1-9, wherein the combined vaccine is used to generate a protective immunity against RSV infection; wherein the first composition is used to elicit an immune response and the second composition is used to boost the immune response; alternatively, the second immunizer is used to elicit the immune response and the first composition is used to boost the immune response.

11. A method for inducing a protective immunity against RSV infection by the combined vaccine according to claim 1, comprising the following steps:
A, administering the first composition to a subject; and
B, administering the second composition to the subject; wherein
step A and step B are conducted in any order.

12. The method according to claim 11, wherein the subject comprises a human or a mouse.

13. The method according to claim 12, wherein when the subject is the mouse, the first composition is administered at a dosage of 1×10¹⁰ vp and the second composition is administered at a dosage of 1×10¹⁰ vp.

14. The method according to claim 11, wherein step A and step B are conducted by intramuscular injection.

15. The combined vaccine according to claim 10, wherein the replication-deficient human adenovirus type 26 vector comprises a nucleotide encoding a pre-fusion fusion glycoprotein (preF) and/or a nucleotide encoding an attachment glycoprotein 130-230aa (G 130-230) of RSV.

16. The combined vaccine according to claim 10, wherein in the second composition, the replication-deficient chimpanzee adenovirus type 63 vector comprises a nucleotide encoding preF and/or a nucleotide encoding G 130-230aa.

17. The combined vaccine according to claim 10, wherein in the second composition, the replication-deficient chimpanzee adenovirus type 63 vector comprises a nucleic acid encoding at least one of three antigenic proteins of RSV.

18. The combined vaccine according to claim 10, wherein in the first composition, the replication-deficient human adenovirus type 26 vector is rAd26; and in the second composition, the replication-deficient chimpanzee adenovirus type 63 vector is rChAd63.

19. A method according to claim 11, wherein the replication-deficient human adenovirus type 26 vector comprises a nucleotide encoding a pre-fusion fusion glycoprotein (preF) and/or a nucleotide encoding an attachment glycoprotein 130-230aa (G 130-230) of RSV.

20. A method according to claim 11, wherein in the second composition, the replication-deficient chimpanzee adenovirus type 63 vector comprises a nucleotide encoding preF and/or a nucleotide encoding G 130-230aa.
